(19) United States
(12) Patent Application Publication
(10) Pub. No.: US 2005/0263430 A1
(43) Pub. Date: Dec. 1, 2005

(54) Container for effervescent tablets

(76) Inventor: Sanso' Giovanni, Milano (IT)

Correspondence Address: YOUNG & THOMPSON, 745 SOUTH 23RD STREET, 2ND FLOOR, ARLINGTON, VA, 22202, US

(21) Appl. No.: 11/134,289

(22) Filed: May 23, 2005

(30) Foreign Application Priority Data
May 24, 2004 (IT) MI2004U000242

(51) Int. Cl. A61K 9/46

(52) U.S. Cl. 206/528; 206/535

(57) ABSTRACT

A container for effervescent tablets 3, includes a tube 2 with a base 7 and upper opening closed by a screw cap 4 provided with a seal in polythene. A spring 10 is placed inside the tube 2 and pushes tablets 3 upwards to facilitate extraction thereof. Guide fins 12, placed at the edge of tube 2, stop tablets 3 at this edge and allow their extraction, one at a time, with a transverse thrust. On base 7 is a tablet of drying agent 8, separated from tablets 3 by a perforated divider 9. At the base of tube 2, a storage unit 13 is attached by screwing, closed by a cap 19, and intended to hold at least one tablet or half a tablet separated by a perforated divider 18 from a drying agent tablet 17 placed on base 16.

The present invention relates to a container, more particularly for effervescent tablets.

Containers for effervescent tablets currently available commercially have a tubular shape and are closed at the top by a cap attached by slotting or screwing.

One disadvantage of these containers is that they do not always allow easy removal of the tablets which at times are jammed between the inner walls of the container, so that the latter has to be turned upside down and shaken vigorously.

Another disadvantage is that the entire container of tablets has to be taken along when it may be necessary to take one tablet or half a tablet away from the place wherein they are normally kept, for example when travelling or at work.

One alternative to this could be that of taking one tablet or half a tablet, according to needs, from the container wherein they are normally contained and wrapping it in a sheet of paper or placing it in another makeshift receptacle. This is not however advisable in that the pill may deteriorate rapidly in that it is no longer in contact with drying agents, normally present on the base of the container or in the respective cap.

The object of the invention is that of eliminating the above disadvantages by providing a container which guarantees easy removal of the tablets and excellent conservation in time of the same.

Another object of the invention is that of providing a container of tablets which allows the user, if necessary, to have available at work or when travelling a tablet or half a tablet of the product without the risk of deterioration of the same.

The container, more particularly for effervescent tablets according to the invention, has the basic features described in the attached independent claim 1.

Preferred embodiments of such a container are disclosed by the dependent claims. Substantially the container according to the invention is formed by a tube in a metal or plastic material suitable for holding divisible effervescent tablets, provided with a cap, preferably screw, with a seal in soft polythene for hermetic closure. A spring is provided inside the container for the upward movement of the tablet and for facilitating extraction thereof without disproportionate manoeuvres being performed.

A fin guide is provided at the upper edge of the tube for stopping the tablets at this edge and allow extraction thereof, one by one, with a simple side thrust.

On the base of the tube a tablet is provided, consisting of a drying agent, such as silica gel or a mixture of both, separated by a perforated divider from the effervescent tablets to allow good conservation of the same.

According to a further feature of the invention, a storage unit is screwed onto the base of the tube, suitable for holding a drying agent tablet, separated by a perforated divider from at least one tablet or half a tablet of the product and closed by a cap in order to have availability thereof when travelling or at work, detaching the storage unit from the main container.

The container according to the invention offers the following advantages:
of a hygienic nature, for extraction of the tablets one at a time;
optimal conservation of the tablets throughout the period of use;
optimal conservation of half tablets once only half thereof has had to be used;
the convenience of the possibility of availability of the quantity necessary for the daily dosage without having to carry along the entire pack.

Additional features of the invention will be made clearer by the following detailed description, given solely as a purely non-limiting embodiment example, illustrated in the accompanying drawings, in which:

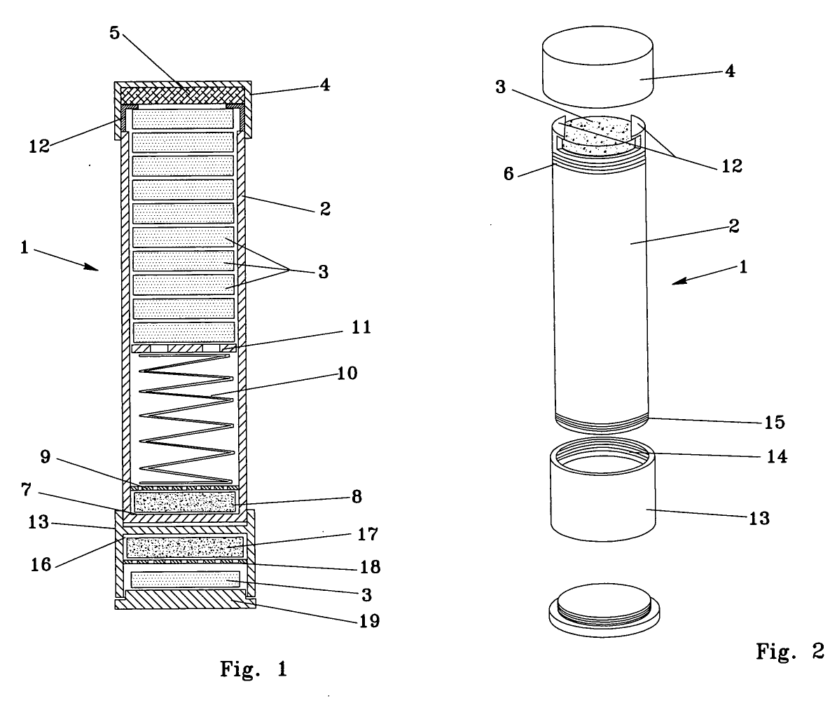

FIG. 1 is an axial section view of the container, more particularly for effervescent tablets according to the invention;

FIG. 2 is a blown-up axonometric view of the container of FIG. 1.

Referring to these drawings, the container, more particularly for divisible effervescent tablets according to the invention, has been denoted overall by reference numeral 1.

It consists of a tube, or main container 2, in a metal or plastic material, containing internally the tablets 3, placed stacked one on the other, and closed at the top by a screw cap 4, provided with a seal in soft polythene 5 to ensure hermetic closure. FIG. 2 schematises the external threading 6 at the upper edge of the tube 2, whereon the cap 4 is screwed.

On the base 7 of the tubular container 2 a pastille 8 of drying agent is placed, such as silica gel or calcium chloride or a mixture of both, suitable for allowing good conservation of the tablets 3 and separated therefrom by a perforated divider 9.

Above the perforated divider 9 a spring 10 is placed which, by means of a perforated disk 11, pushes the tablets 3 upwards to allow easy extraction thereof without having to perform complicated manoeuvres.

At the upper edge of the tube 2 a pair of guide fins 12 are provided which stop the tablets 3 at the edge of the tube when the container is closed (FIG. 1) and project externally from this edge following removal of the cap, in such a way as to allow extraction of the tablets one at a time with a simple transverse thrust (FIG. 2).

A storage unit 13 is attached at the lower end of the tube 2, appropriately by screwing. FIG. 2 schematises an internal threading 14 of the storage unit and external threading 15 of the tube 2.

The storage unit 13 contains on its base 16, placed adjacent to the base 7 of the tube 2, a tablet 17 of drying material, separated by a perforated divider 18 from a tablet 3 or half a tablet of the effervescent product.

The storage unit 3 is tightly closed by a cap 19.

Naturally the storage unit 13 can be dimensioned so as to contain more than one tablet 3, should the daily dosage require the taking of more than one tablet. In this case the user can remove the storage unit 3 from the tube 1 to have availability of the quantity of product necessary for the daily dosage, without having to take along the entire container 1.

From what has been shown, the advantages of the container specifically for effervescent tablets according to the invention are clear.

The invention is not however limited to the particular form of the embodiment previously described and illustrated in the accompanying drawings, but instead a number of detail changes may be made thereto, within the scope of a person skilled in the art, without thereby departing from the sphere of the invention, defined by the following claims.

1. Container for tablets 3 comprising a tube 2 provided with a base 7 and closed at the top by a cap 4, characterised in that inside said tube 2 a spring 10 is placed, suitable for pushing the tablets towards the end of the tube closed by said cap 4, to facilitate extraction thereof.

2. Container according to claim 1, characterised in that at the edge of the tube 2 guide fins 12 are provided, suitable for stopping the tablets 3 at this edge when the cap 4 is attached on the tube 2, and for projecting from this edge when the cap 4 is removed, so as to allow extraction of the tablets 4, one at a time, by a simple transverse thrust.

3. Container according to claim 1, characterised in that said cap 4 is screw-attached onto the tube 2.

4. Container according to claim 1, characterised in that said cap 4 is

5. Container according to claim 1, characterised in that on said base 7 of the tube 2 a tablet 8 of drying agent, such as silica gel or calcium chloride or a mixture of both, is placed, separated by a perforated divider 9 from said tablets 3 of product.

6. Container according to claim 1, characterised in that on the base of said tube 2 a storage unit 13 is attached, tightly closed by a cap 19 and suitable for containing at least one tablet or half a tablet 3 of product.

7. Container according to claim 6, characterised in that said storage unit 13 is attached to the tube 2 by screwing.

8. Container according to claim 6, characterised in that said storage unit 13 contains on its base 16, suitable for positioning adjacent to said base 7 of the tube 2, a tablet 17 of drying agent, separated from said one at least tablet or half tablet 3 of product by a perforated divider 18.

9. Container according to claim 1, characterised in that its components are made in a metal material, plastic material or both.
